(19) United States
(12) Patent Application Publication
(10) Pub. No.: US 2008/0294002 A1
(43) Pub. Date: Nov. 27, 2008

(54) ENDOSCOPE APPARATUS AND ENDOSCOPE PROBE

(75) Inventor: Tianyu XIE, Beijing (CN)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 11/842,559

(22) Filed: Aug. 21, 2007

(30) Foreign Application Priority Data
Aug. 22, 2006 (JP) 2006-225786

(51) Int. Cl. A61B 1/04; A61B 1/06

(52) U.S. Cl. 600/109; 600/160

(57) ABSTRACT

An endoscope apparatus of the invention is provided with an endoscope insertion portion to be inserted into a body cavity and is capable of performing fluorescence observation. The endoscope apparatus includes: a transferring portion, which is provided to the endoscope insertion portion, for transferring information on an image of a region to be examined to a proximal end side of the endoscope insertion portion; a light deflecting portion for directing fluorescence entered from a side surface of a distal end portion of the endoscope insertion portion to one end side of the transferring portion; and an image forming optical system for image-forming fluorescence from the light deflecting portion at one end of the transferring portion.

This application claims benefit of Japanese Application No. 2006-225786 filed in Japan on Aug. 22, 2006, the contents of which are incorporated by this reference.

### BACKGROUND OF THE INVENTION

1. Field of the Invention

The present invention relates to an endoscope apparatus capable of performing fluorescence observation in which a living body is irradiated with excitation light and fluorescence from a lesioned part is received.

2. Description of the Related Art

Conventionally, an endoscope apparatus has been widely used in a medical field and the like. In particular, the endoscope apparatus in the medical field is used for examination, observation, and the like in a living body as a subject. One example of such endoscope observation is fluorescence observation in which an image is generated by irradiating the living body with excitation light and receiving fluorescence from the lesioned part. When such fluorescence observation is performed, a fluorescence material such as hematoporphyrin having an affinity for the lesioned part such as carcinoma is administered to a patient's body in advance. In the fluorescence observation, after a predetermined time period has passed after the administration of the fluorescence material, a fluorescence image is obtained by irradiating the lesioned part with excitation light and receiving fluorescence generated from the fluorescence material accumulated on the lesioned part, and a state of the lesioned part is judged by the fluorescence image.

An endoscope apparatus performing such fluorescence observation is disclosed, for example, in Japanese Patent Unexamined Publications No. 10-151104 and No. 10-201707. The endoscope apparatuses disclosed in these publications are capable of performing the fluorescence observation by irradiating the excitation light to the living tissue of the lesioned part from a distal end portion of an endoscope insertion portion, and using a fluorescence image obtained by receiving fluorescence from the living tissue.

In the conventional endoscope apparatus, an excitation filter for transmitting only excitation light having a desired band is disposed on a light-emission side of an excitation light source, and a barrier filter for transmitting only fluorescence having a desired band is disposed on light-incident side of a solid image pickup element.

### SUMMARY OF THE INVENTION

An endoscope apparatus of the present invention is provided with an endoscope insertion portion to be inserted into a body cavity and is capable of performing fluorescence observation. The endoscope apparatus comprises: a transferring portion, which is provided to the endoscope insertion portion, for transferring information on an image of a region to be examined to a proximal end side of the endoscope insertion portion; a light deflecting portion for directing fluorescence entered from a side surface of a distal end portion of the endoscope insertion portion to one end side of the transferring portion; and an image forming optical system for image-forming fluorescence from the light deflecting portion at one end of the transferring portion.

Furthermore, an endoscope probe of the present invention is provided with a probe insertable into a treatment instrument insertion channel of the endoscope insertion portion to be inserted into a body cavity and is capable of performing fluorescence observation. The endoscope probe comprises: a transferring portion, which is provided to the probe, for transferring information on an image of a region to be examined to a proximal end side of the probe; a light deflecting portion for directing fluorescence entered from a side surface of a distal end portion of the probe to one end side of the transferring portion; and an image forming optical system for image-forming fluorescence from the light deflecting portion at one end of the transferring portion.

The above and other features and advantages of the present invention will be more clearly understood from the following description.

### BRIEF DESCRIPTION OF THE DRAWINGS

FIGS. 1 to 10 relate to a first embodiment of the present invention in which:

FIG. 1 is an overall configurational view showing a schematic configuration of an endoscope apparatus of the first embodiment;

FIG. 2 is a front view of an endoscope insertion portion showing a modification example of a distal end portion of FIG. 1;

FIG. 3 is an explanatory view showing a working of the endoscope insertion portion of FIG. 1;

FIG. 4 is an explanatory view showing a monitor display example;

FIG. 5 is an explanatory view showing a first modification example of the monitor display example;

FIG. 6 is an explanatory view showing a second modification example of the monitor display example of FIG. 4;

FIG. 7 is an overall configurational view showing a modification example of the endoscope apparatus of FIG. 1;

FIG. 8 is a front view of an endoscope insertion portion of FIG. 7;

FIG. 9 is an explanatory view showing a graph of fluorescence intensity with respect to a distance, which is obtained by the endoscope apparatus of FIG. 7; and

FIG. 10 is a front view of an endoscope insertion portion showing a modification example of a distal end portion of FIG. 8.

FIGS. 11 to 14 relate to a second embodiment of the present invention in which:

FIG. 11 is an overall configurational view showing a schematic configuration of the endoscope apparatus of the second embodiment;

FIG. 12 is a front view of a fluorescence probe of FIG. 11;

FIG. 13 is a first explanatory view showing a schematic configuration of the fluorescence probe of FIG. 11; and

FIG. 14 is a second explanatory view showing a schematic configuration of the fluorescence probe of FIG. 11.

FIGS. 15 to 20 relate to modification examples of the second embodiment of the present invention in which:

FIG. 15 is a view showing a schematic configuration of a fluorescence probe according to a first modification example;

FIG. 16 is a perspective view showing a cone mirror provided to a transparent tube of FIG. 15;

FIG. 17 is a view showing a test sample to be examined which will be photographed by the endoscope apparatus provided with the fluorescence probe of FIG. 15;

FIG. 18 is a view showing a photographed image of the test sample to be examined of FIG. 17;

FIG. 19 is a view showing a schematic configuration of a fluorescence probe according to a second modification example; and

FIG. 20 is a view showing a schematic configuration of a fluorescence probe according to a third modification example.

### DETAILED DESCRIPTION OF THE PREFERRED EMBODIMENTS

### First Embodiment

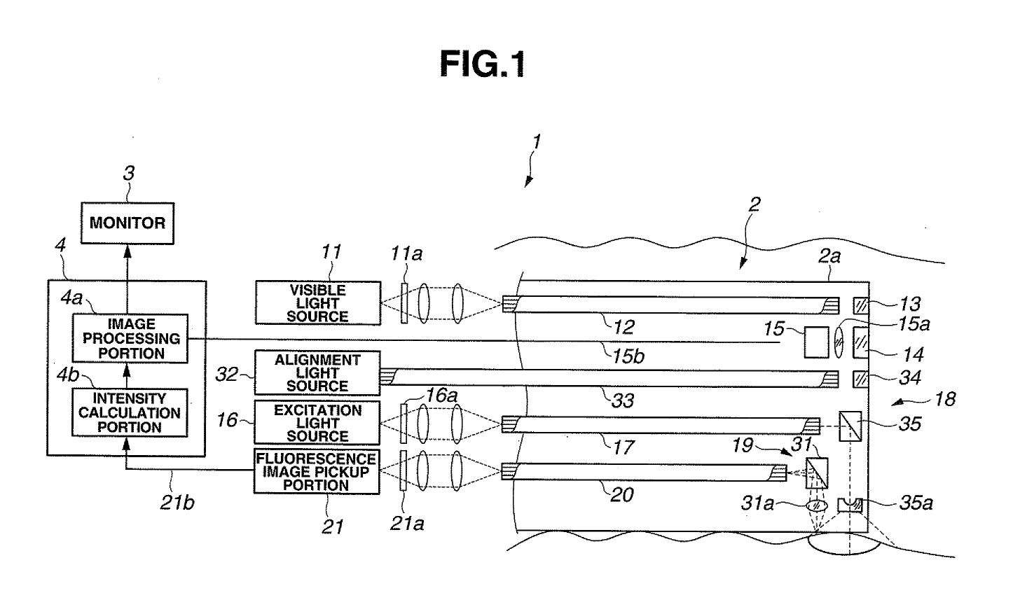

As shown in FIG. 1, an endoscope apparatus 1 of the first embodiment includes an elongated endoscope insertion portion 2 to be inserted into a body cavity, and an image processing apparatus 4 for displaying on a monitor 3 an endoscope image obtained by processing an image pickup signal generated by the white light image pickup portion, to be described later, of the endoscope insertion portion 2.

The endoscope insertion portion 2 is connected to a white light source 11 from which visible light such as white light is supplied to the endoscope insertion portion 2. A visible light filter 11a is disposed on a light-emission side of the white light source 11. The visible light filter 11a transmits visible light having a desired band. Note that, in a case of a frame-sequential type, the visible light filter 11a may be a rotation filter which rotates an RGB filter for transmitting R (red), G (green), and B (blue) lights every predetermined time period.

The visible light supplied to the endoscope insertion portion 2 is transmitted to a distal end portion 2a by a white light guide 12 as a visible light transmitting portion which is inserted in the endoscope insertion portion 2. Then, the visible light illuminates a living tissue of a target region via a white optical system 13 provided to the distal end portion 2a.

Reflected light from the illuminated living tissue is taken in as a subject image by an objective optical system 15a trough an observation window 14 provided to the distal end portion 2a of the endoscope insertion portion 2. At an image-forming position of the objective optical system 15a, is provided a white light image pickup portion 15 including a solid image pickup element such as a CCD (Charge Coupled Device) or a CMOS (Complementary Metal Oxide Semiconductor). Then the white light image pickup portion 15 picks up the subject image. The white light image pickup portion 15 photoelectrically converts the subject image to generate an image pickup signal. Then, the white light image pickup portion 15 outputs the generated image pickup signal to the image processing apparatus 4 via a signal cable 15b serving as a transferring portion for transferring information on an image of a region to be examined.

The image processing apparatus 4 processes the image pickup signal by the image processing portion 4a to generate a standard video signal. The image processing apparatus 4 outputs the video signal to the monitor 3 to display the endoscope image on a display screen of the monitor 3.

In addition, the endoscope insertion portion 2 is connected to an excitation light source 16. Then, excitation light such as laser light is supplied to the endoscope insertion portion 2 from the excitation light source 16. On a light-emission side of the excitation light source 16, an excitation light filter 16a is disposed. Then, the excitation light filter 16a transmits the excitation light having a desired band.

The excitation light supplied to the endoscope insertion portion 2 is transmitted to the distal end portion 2a by an excitation light guide 17 as an excitation light transmitting portion, which is inserted in the endoscope insertion portion 2. Then, the excitation light is irradiated to the living tissue of the target region via an excitation optical system 18 as an excitation light irradiating portion, which is provided to the distal end portion 2a.

The living tissue (fluorescence material included therein) irradiated with excitation light generates fluorescence (photoluminescence or cold light), and the fluorescence is taken in by a fluorescence optical system 19 as a fluorescence-intake portion provided at the distal end portion 2a of the endoscope insertion portion 2. At an image-forming portion of the fluorescence optical system 19, is disposed an incident end of a fluorescence image guide 20 as a fluorescence-transmitting portion (transferring portion), which is inserted in the endoscope insertion portion 2, for transferring information on an image of the region to be examined. The fluorescence image guide 20 transmits fluorescence entered from the incident end thereof to an emission end on a proximal end side of the insertion portion, to emit the transmitted fluorescence to a fluorescence image pickup portion 21 having a solid image pickup element such as a CCD or a CMOS, which is provided to the proximal end (emission end) side.

On an incident side of the fluorescence image pickup portion 21, is disposed a barrier filter 21a through which fluorescence having a desired band is passed. The fluorescence image pickup portion 21 photoelectrically converts the picked-up image of the fluorescence to generate an electrical signal. Then the fluorescence image pickup portion 21 outputs the generated electrical signal to the image processing apparatus 4 via the signal cable 15b.

The image processing apparatus 4 calculates intensity of the fluorescence in an intensity calculation portion 4b based on the electrical signal outputted from the fluorescence image pickup portion 21 to generate calculation data, and outputs the calculation data to the image processing portion 4a. The image processing portion 4a performs, based on the calculation data from the intensity calculation portion 4b, image display processing for displaying fluorescence intensity together with the endoscope image as described later, to display the images obtained by the processing on a display screen of the monitor 3.

Note that, though the fluorescence image pickup portion 21 is disposed at the emission end of the fluorescence image guide 20 in the present embodiment, the fluorescence image pickup portion 21 and the intensity calculation portion 4b may be electrically connected by disposing the fluorescence image pickup portion 21 at an image forming position of the fluorescence optical system 19 and disposing a signal cable (signal line) so as to be inserted through the endoscope insertion portion 2.

In addition, the excitation light filter 16a and the barrier filter 21a are configured to transmit only the light having a desired band. However, the actual filters also transmit the light having a band near the desired band. As a result, a part of the light passed through the excitation light filter 16a becomes leak light. The leak light is generated by a part of reflected light of the excitation light reflected from a normal tissue and a lesioned part, and the leak light is fainter than the fluorescence generated from the lesioned part.

However, intensity of light is proportional to a square of the reciprocal number of the distance, so that the intensity of the fluorescence from the lesioned part existing far from the distal end portion 2a of the endoscope insertion portion 2 is smaller than that of the leak light generated by a part of the reflected light of the excitation light reflected from the normal tissue existing in the vicinity of the distal end portion 2a of the endoscope insertion portion 2.

Even if light leakage occurs from the excitation light filter 16a and the barrier filter 21a, it is possible to always distinguish the normal tissue from the lesioned part in the present embodiment. More specifically, the fluorescence optical system 19 includes a fluorescence prism 31 as a light deflecting portion provided on a side surface of the distal end portion 2a of the endoscope insertion portion 2 and an image forming optical system 31a for allowing the fluorescence from the lesioned part existing on an inner wall of the body cavity and the reflected light of the excitation light from the lesioned part and the normal tissue to enter the fluorescence prism 31. Accordingly, the fluorescence optical system 19 is capable of taking the light in from the side surface of the distal end portion 2a of the endoscope insertion portion 2 using the fluorescence prism 31 and the image forming optical system 31a.

Therefore, the endoscope insertion portion 2, by disposing the distal end portion 2a generally parallel with the inner wall of the body cavity as described later, can take in the fluorescence from the lesioned part existing on the inner wall of the body cavity in the observation field of view and the reflected light from the normal tissue at approximately the same distance, and can direct the fluorescence and the reflected light such that the images thereof are formed at the incident end of the fluorescence image guide 20.

Note that, to the endoscope insertion portion 2, is connected an alignment light source 32 for generating, for example, blue alignment light to show a position of the fluorescence optical system 19 on the display screen of the monitor 3. The alignment light supplied from the alignment light source 32 is transmitted to the distal end portion 2a by an alignment light guide 33 inserted through the endoscope insertion portion 2, to be emitted in the body cavity via an alignment optical system 34 as an alignment light irradiating portion provided at the distal end portion 2a.

The image of the reflected light generated by reflection of the alignment light from inside of the body cavity is picked up by the white light image pickup portion 15 to be displayed on the endoscope image, as described later Note that the alignment optical system 34 is disposed on a side closer to the fluorescence optical system 19 of the distal end portion 2a, and the position of the fluorescence optical system 19 can be confirmed on the endoscope image.

Note that the alignment light is supplied from the alignment light source 32 to the endoscope insertion portion 2 to be transmitted to the distal end portion 2a by the alignment light guide 33, and is emitted into the body cavity via the alignment optical system 34 in the present embodiment. However, an LED (Light Emitting Diode) may be provided to the distal end portion 2a as the alignment light source 32 to directly emit the alignment light into the body cavity from the LED.

Meanwhile, similarly as the fluorescence optical system 19, the excitation optical system 18 also includes an excitation light prism 35 provided on the side surface of the distal end portion 2a of the endoscope insertion portion 2, and an excitation light lens 35a for spreading out the excitation light from the excitation light prism 35. Accordingly, the excitation optical system 18 is capable of directing the excitation light in a radially outward direction of the endoscope insertion portion 2 by the excitation light prism 35 and the excitation light lens 35a and irradiating the excitation light to the inner wall of the body cavity from the side surface of the distal end portion 2a of the endoscope insertion portion 2.

The fluorescence optical system 19 and the excitation optical system 18 are adjoiningly disposed. In the present embodiment, the fluorescence optical system 19 and the excitation optical system 18 are disposed before and behind each other along a longitudinal axis direction of the endoscope insertion portion 2.

Note that, in the present embodiment, the excitation light is supplied from the excitation light source 16 to the endoscope insertion portion 2, to be transmitted to the distal end portion 2a by the excitation light guide 17 and emitted to the inner wall of the body cavity via the excitation optical system 18. However, an LED may be provided as the excitation light source 16 on the side surface of the distal end portion 2a of the endoscope insertion portion 2 to directly emit the excitation light to the inner wall of the body cavity from the LED.

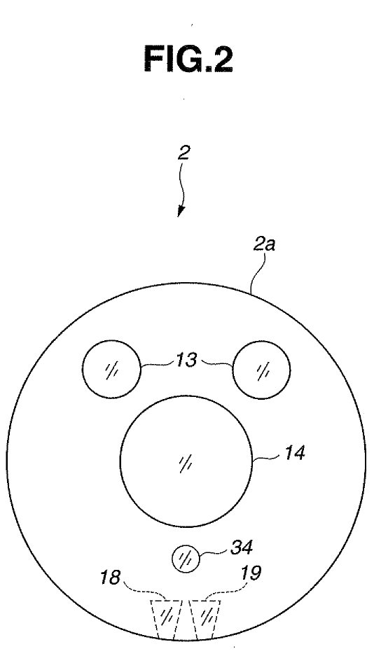

In addition, as shown in FIG. 2, the fluorescence optical system 19 and the excitation optical system 18 may be adjoiningly disposed in a circuit direction of the distal end portion 2a.

In the endoscope apparatus 1 configured as such, as shown in FIG. 1, the endoscope insertion portion 2 is connected to the various light sources, the fluorescence image pickup portion 21, and the image processing apparatus 4, to be inserted into the body cavity, and the distal end portion 2a is guided to a target region, thereafter fluorescence observation is performed. Note that the target region is assumed to be a lesioned part existing in the small intestine or the duodenum in the present embodiment.

First, an operator administers a fluorescence material such as hematoporphyrin, for example, into a patient's body in advance. After a predetermined time period has passed, the fluorescence material is accumulated on the lesioned part. The operator inserts the endoscope insertion portion 2 into the body cavity of the patient, and guides the distal end portion 2a to the target region.

At this time, in the endoscope insertion portion 2, visible light such as white light supplied from the white light source 11 passes through the white light guide 12 to illuminate the living tissue in the body cavity via the white optical system 13. The endoscope insertion portion 2 takes in the reflected light from the illuminated living tissue by the objective optical system 15a as a subject image through the observation window 14, and picks up the subject image by the white light image pickup portion 15. The white light image pickup portion 15 photoelectrically converts the subject image to generate an image pickup signal, and then outputs the generated image pickup signal to the image processing apparatus 4.

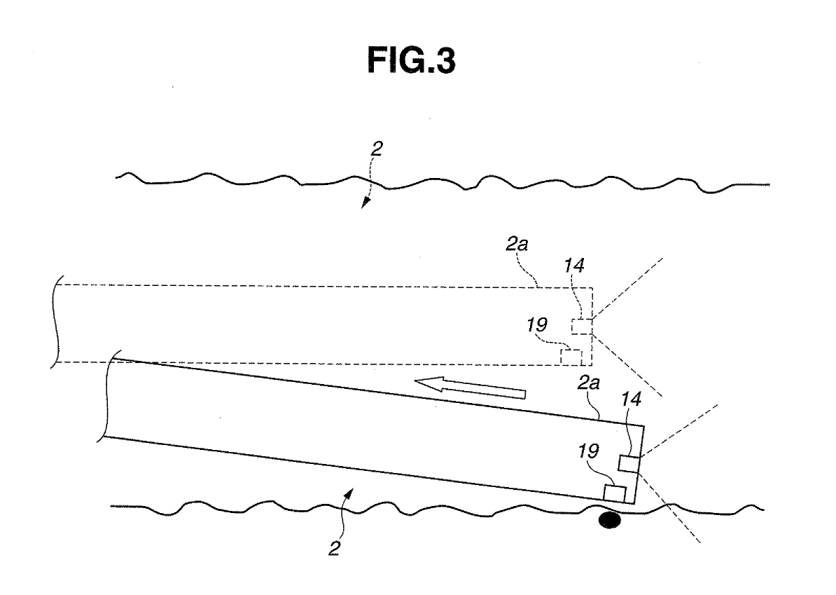

The image processing apparatus 4 processes the image pickup signal by the image processing portion 4a to generate a standard video signal, and outputs the video signal to the monitor 3 to display an endoscope image on the monitor 3. The operator guides the distal end portion 2a of the endoscope insertion portion 2 into the target region in the body cavity, while watching the display screen of the monitor 3. As shown by a dotted line in FIG. 3, when the distal end portion 2a of the endoscope insertion portion 2 reaches the target region, the operator performs fluorescence observation.

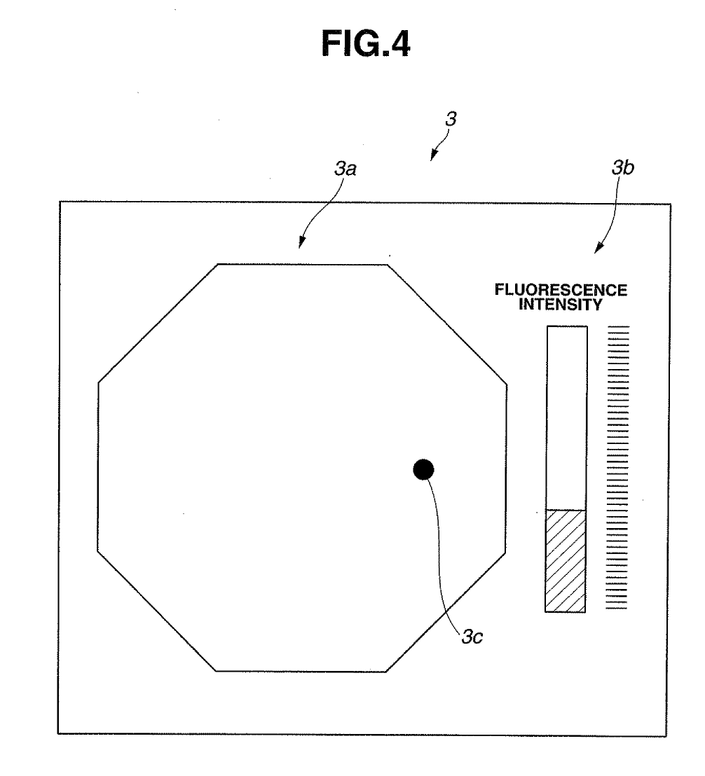

First, the operator irradiates inside of the body cavity with the alignment light in order to confirm the position of the fluorescence optical system 19 on the endoscope image. In the endoscope insertion portion 2, the alignment light from the alignment light source 32 passes through the alignment light guide 33 to be emitted into the body cavity via the alignment optical system 34. The image of the reflected light generated by the reflection of the alignment light from inside the body cavity is picked up by the white light image pickup portion 15 and displayed on the endoscope image (see FIG. 4). The operator confirms the position of the fluorescence optical system 19 by alignment light 3c displayed on the endoscope image. The operator locates the distal end portion 2a such that the side surface thereof on which the fluorescence optical system 19 is provided is approximately parallel with the inner wall of the body cavity, as shown by a solid line in FIG. 3. This makes it possible for the endoscope insertion portion 2 to take in the fluorescence from the lesioned part existing on the inner wall of the body cavity and the reflected light from the normal tissue, from the fluorescence optical system 19 at approximately the same distance.

Next, the operator supplies the excitation light to the endoscope insertion portion 2 to start performing fluorescence observation.

The operator moves the distal end portion 2a of the endoscope insertion portion 2 forwardly and backwardly along the inner wall of the body cavity to perform fluorescence observation of the vicinity of the target region. Note that FIG. 3 shows a state where the distal end portion 2a of the endoscope insertion portion 2 is being moved backwardly in an arrow direction.

In the endoscope insertion portion 2, the excitation light from the excitation light source 16 passes through the excitation light guide 17 via the excitation light filter 16a, to be irradiated to the living tissue on the inner wall of the body cavity from the side surface of the distal end portion 2a via the excitation optical system 18. When a lesioned part exists in the living tissue on the inner wall of the body cavity irradiated with the excitation light, the living tissue of the lesioned part (fluorescence material included therein) generates fluorescence. At this time, also the excitation light is irradiated to the lesioned part and the normal tissue to be reflected therefrom.

The fluorescence from the lesioned part and the reflected light from the lesioned part and the normal tissue are taken in via the fluorescence optical system 19. This makes it possible for the endoscope insertion portion 2 to take in the fluorescence from the lesioned part existing on the inner wall of the body cavity and the reflected light from the normal tissue at approximately the same distance.

The fluorescence from the lesioned part and the reflected light from the lesioned part and the normal tissue which are taken in by the fluorescence optical system 19 are transmitted by the fluorescence image guide 20, and images thereof are picked up by the fluorescence image pickup portion 21 via the barrier filter 21a. The fluorescence image pickup portion 21 generates an electrical signal by photoelectrically converting the picked-up image of the fluorescence, to output the generated electrical signal to the image processing apparatus 4 via a signal line 21b.

The image processing apparatus 4 outputs to the image processing portion 4a the calculation data obtained by calculating the fluorescence intensity by the intensity calculation portion 4b based on the electrical signal from the fluorescence image pickup portion 21. The image processing portion 4a performs an image display processing based on the calculation data from the intensity calculation portion 4b to output the video signal obtained by the image display processing to the monitor 3.

As shown in FIG. 4, a fluorescence intensity display portion 3b is provided on the right side of the endoscope image display portion 3a on the display screen of the monitor 3, and the fluorescence intensity is displayed together with the endoscope image. Accordingly, the endoscope apparatus 1 can quantitatively measure the fluorescence intensity.

As described above, when the operator moves the distal end portion 2a of the endoscope insertion portion 2 forwardly and backwardly along the inner wall of the body cavity, the calculated fluorescence intensity is displayed on the fluorescence intensity display portion 3b depending on the forward/backward movement. Therefore, the position where the fluorescence intensity is strong can be identified as a position where the lesioned part exists.

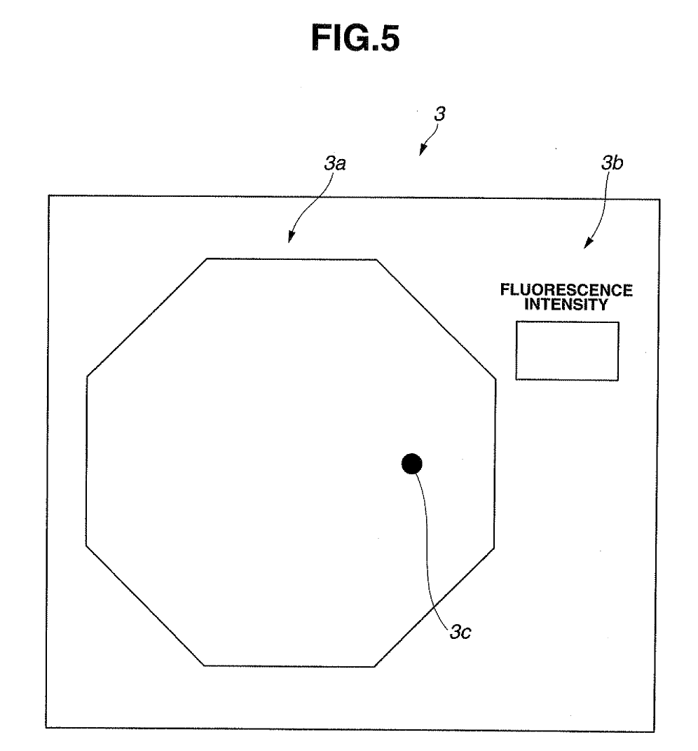

Note that though the fluorescence intensity shown in FIG. 4 is displayed as a bar, the fluorescence intensity may be displayed as a numerical value as shown in FIG. 5. As shown in FIG. 5, the numerical value of the calculated fluorescence intensity is displayed on the fluorescence intensity display portion 3b.

As a result, in the endoscope apparatus 1 of the present embodiment, the endoscope insertion portion 2 can take in the fluorescence from the lesioned part existing on the inner wall of the body cavity and the reflected light from the normal tissue, from the fluorescence optical system 19 at approximately the same distance. This makes it possible to always distinguish the normal tissue from the lesioned part, since the intensity of the fluorescence from the lesioned part existing further from the distal end portion 2a of the endoscope insertion portion 2 does not become weaker, compared with that of the leak light of the reflected light from the normal tissue existing nearer to the distal end portion 2a.

Note that in the endoscope apparatus 1, the position of the fluorescence optical system 19 may be shown by image processing without using the alignment light 3c. In this case, the image processing portion 4a of the image processing apparatus 4 displays the image indicating the position of the fluorescence optical system 19 depending on the direction of the endoscope insertion portion 2, based on direction information from a direction detecting portion, not shown, for detecting the direction of the endoscope insertion portion 2.

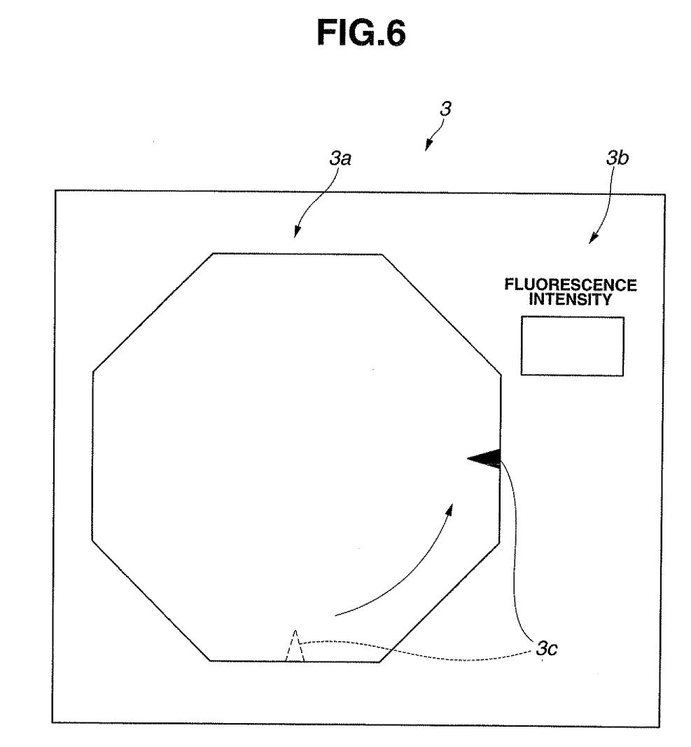

As shown in FIG. 6, on the endoscope image in the endoscope image display portion 3a, a black triangle 3d is displayed as a marking indicating the position of the fluorescence optical system 19 depending on the direction of the endoscope insertion portion 2. This configuration eliminates the need for providing the alignment light source 32, the alignment light guide 33, and the alignment optical system 34 to the endoscope insertion portion 2, thereby allowing the reduction of the diameter of the endoscope insertion portion 2.

Note that it may be configured such that the fluorescence optical system 19 and the fluorescence image guide 20 may also serve as the excitation optical system 18 and the excitation light guide 17, respectively.

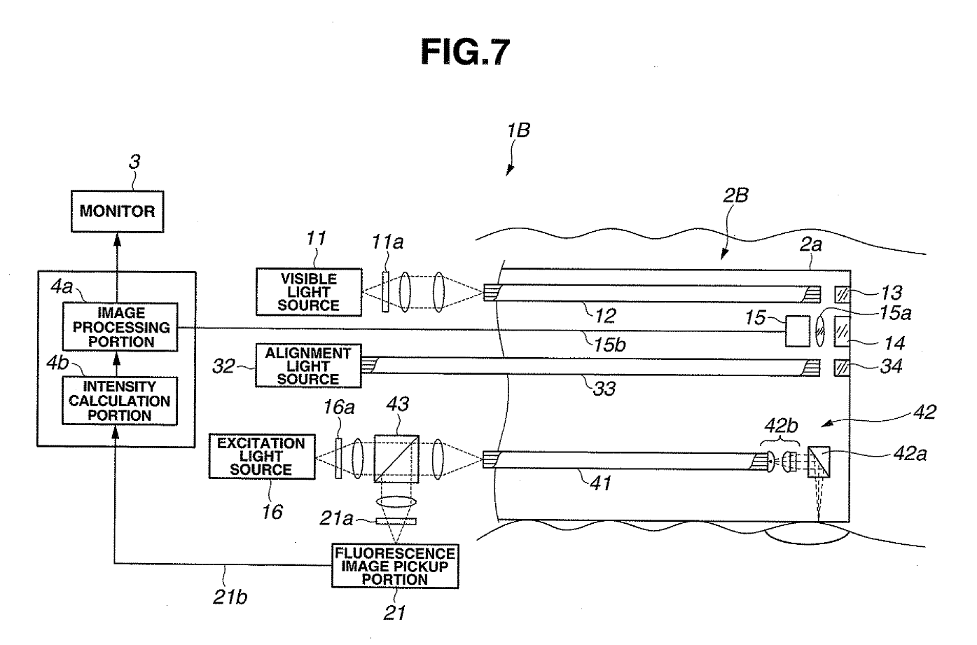

As shown in FIG. 7, the endoscope apparatus 1B includes an endoscope insertion portion 2B having inserted thereto a multi-purpose image guide 41 as a fluorescence transmitting portion (transferring portion) for transferring information on an image of a region to be examined, serving both as the fluorescence image guide 20 and the excitation light guide 17 and having on the side surface of the distal end portion 2a a multi-purpose optical system 42 serving both as the fluorescence optical system 19 and the excitation optical system 18. The multi-purpose optical system 42 includes a multi-purpose prism 42a and an irradiation/image forming optical system 42b. That is, the multi-purpose optical system 42 functions as an excitation light irradiating portion for irradiating the excitation light emitted from the excitation light guide 17 in a radially outward direction of the endoscope insertion portion 2B. In addition, the multi-purpose prism 42a and the irradiation/image formation optical system 42b function as a deflection optical system and image forming optical system, respectively.

On the proximal end side of the multi-purpose image guide 41, a dichroic prism 43 (or a dichroic mirror) is provided, so that an optical path is diverged into two directions, that is, one is toward the excitation light source 16 and the other is toward the fluorescence image pickup portion 21. With such a configuration, the excitation light from the excitation light source 16 enters the dichroic prism 43 via the excitation light filter 16a, and is transmitted from the dichroic prism 43 to the multi-purpose image guide 41, to be irradiated from the side surface of the distal end portion 2a, via the multi-purpose optical system 42, to the living tissue on the inner surface of the body cavity.

The light from the lesioned part and the reflected light from the normal tissue are taken in from the side surface of the distal end portion 2a via the multi-purpose optical system 42, and is transmitted through the multi-purpose image guide 41 to enter the dichroic prism 43 by which fluorescence components generated from the lesioned part is reflected, and the images of the fluorescence components are picked up by the fluorescence image pickup portion 21 via the barrier filter 21a.

As a result, the endoscope apparatus 1B employs the multi-purpose image guide 41 and the multi-purpose optical system 42, thereby allowing a diameter smaller than that of the endoscope insertion portion 2.

Note that the fluorescence image guide 20 and the excitation light guide 17 may be provided in plural numbers.

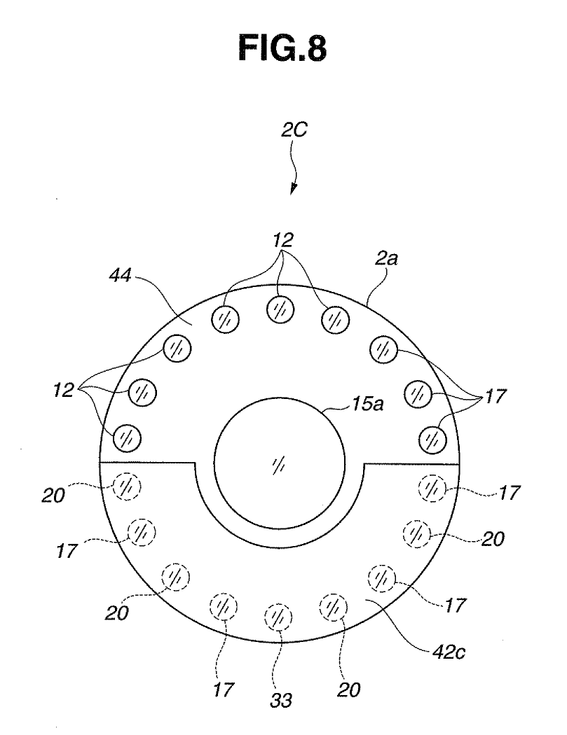

As shown in FIG. 8, the endoscope apparatus includes an endoscope insertion portion 2C having a plurality of fluorescence image guides 20 and excitation light guides 17 inserted in a circuit direction over an approximately half-periphery thereof (lower half in FIG. 8). On the side surface of the distal end portion 2a of the endoscope insertion portion 2C, there is provided a multi-purpose prism 42c covering approximately the half-periphery of the distal end portion 2a as a multi-purpose optical system serving both as the fluorescence optical system 19 and the excitation optical system 18.

In addition, the endoscope insertion portion 2C includes a plurality of white light guides 12 inserted therein over an approximately half-periphery opposite to the half-periphery where the fluorescence image guides 20 and the excitation light guides 17 are disposed (upper half in FIG. 8).

At the distal end portion 2a of the endoscope insertion portion 2C, an observation window 44, as the white optical system 13, which transmits visible light emitted from the plurality of light guides 12 and transmits the visible light reflected from inside of the body cavity to the white light image pickup portion 15, is provided so as to cover the half-periphery of the distal end portion 2a.

With such a configuration, the endoscope insertion portion 2C illuminates inside of the body cavity with the visible light from the plurality of white light guides 12 through the observation window 44 and takes in, through the observation window 44, the reflected light of the visible light from inside of the illuminated body cavity as a subject image by the objective optical system 15a, to pick up the subject image by the white light image pickup portion 15.

In addition, the endoscope insertion portion 2C irradiates, via the multi-purpose prism 42c, the excitation light emitted from the plurality of excitation light guides 17 to the living tissue on the inner wall of the body cavity from around the half-periphery of the side surface of the distal end portion 2a, and takes in, via the multi-purpose prism 42c, the fluorescence from the lesioned part and the leak light from the normal tissue from around the half-periphery of the side surface of the distal end portion 2a, to introduce the fluorescence and the leak light to the plurality of fluorescence image guides 20. The image of the fluorescence transmitted by each of the plurality of fluorescence image guides 20 is picked up respectively by the fluorescence image pickup portion 21.

The fluorescence image pickup portion 21 photoelectrically converts the respectively picked-up image of the fluorescence to generate an electrical signal, and outputs the generated electrical signal to the image processing apparatus 4 via the signal line 21b. The image processing apparatus 4 outputs to the image processing portion 4a calculation data obtained by calculating the intensity of the fluorescence in the intensity calculation portion 4b based on the electrical signal from the fluorescence image pickup portion 21. The image processing portion 4a performs image display processing based on the calculation data from the intensity calculation portion 4b to output the video signal obtained by the image display processing to the monitor 3.

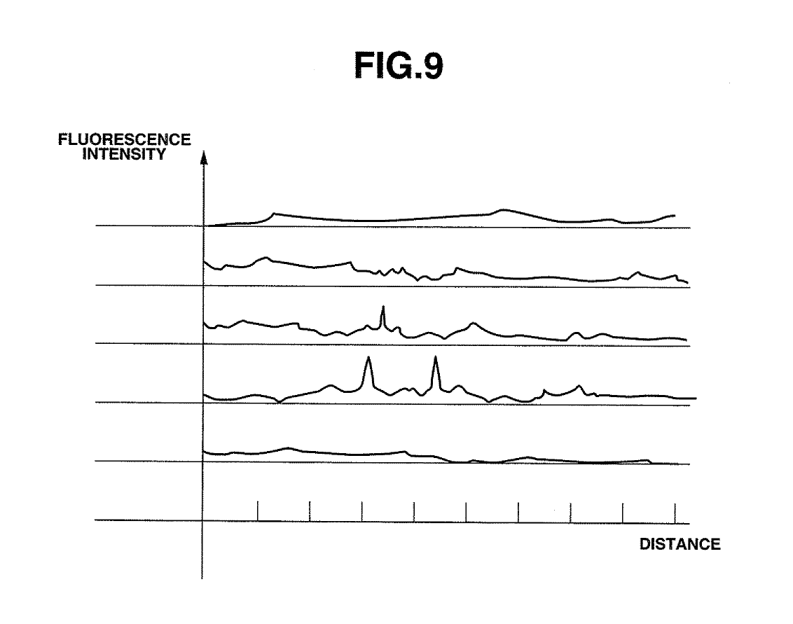

The image display processing may be performed in such a manner that an image of the fluorescence is respectively generated depending on each of the plurality of fluorescence image guides 20. In this case, as shown in FIG. 9, a graph is displayed to show the fluorescence intensities with respect to forward/backward movement distance (time period) of the distal end portion 2a for each of the plurality of fluorescence image guides 20.

According to this, the endoscope insertion portion 2C is capable of irradiating the excitation light to the inner wall of the body cavity from over the half-periphery of the distal end portion 2a, and also capable of taking in the fluorescence from over the half-periphery of the distal end portion 2a, thereby allowing fluorescence observation with respect to a wider range of the inner wall of the body cavity.

Note that the excitation light guide 17 and the fluorescence image guide 20 may be the multi-purpose image guide 41 serving as both of them, similarly as described in FIG. 7.

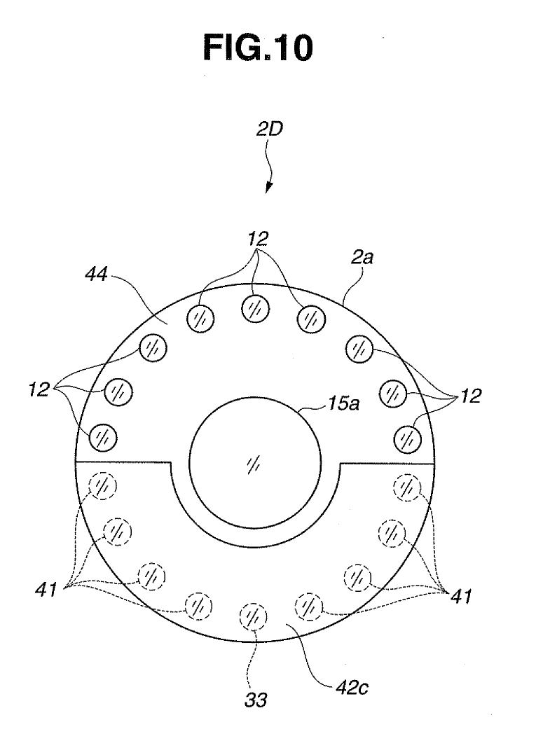

As shown in FIG. 10, an endoscope insertion portion 2D includes inserted therein the multi-purpose image guide 41 serving both as the excitation light guide 17 and the fluorescence image guide 20.

This enables the endoscope insertion portion 2D to perform more minute fluorescence observation compared with the endoscope insertion portion 2C.

Note that the present embodiment is so configured as to calculate the fluorescence intensity by the intensity calculation portion 4b based on the electrical signal generated by picking up the image of the fluorescence in the fluorescence image pickup portion 21 to display the calculated fluorescence intensity. However, the present invention is not limited to the same, and the electrical signal generated in the fluorescence image pickup portion 21 may be displayed as it is, as the fluorescence intensity.

### Second Embodiment

In the first embodiment, the fluorescence optical system 19 as the fluorescence-intake portion is disposed on the side surface of the distal end portion 2a of the endoscope insertion portion. In the second embodiment, the fluorescence optical system 19 as the fluorescence-intake portion is disposed on a side surface of a probe distal end portion of a probe which is insertable through a treatment instrument insertion channel of the endoscope insertion portion. Other configurations are the same as those in the above-described first embodiment, so that descriptions thereof are omitted, and the same components will be described by attaching the same reference symbols.

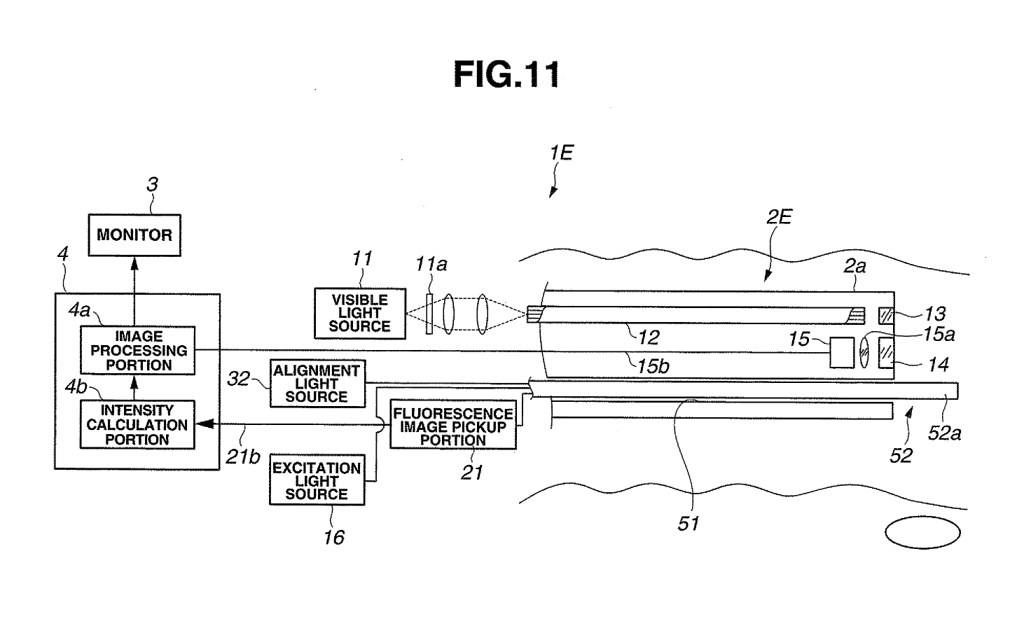

That is, as shown in FIG. 11, an endoscope apparatus 1E of the second embodiment includes a fluorescence probe 52 inserted through a treatment instrument insertion channel 51 of an endoscope insertion portion 2E. Note that on a proximal end side of the endoscope insertion portion 2E, a treatment instrument insertion port, not shown, communicating with the treatment instrument insertion channel 51 is formed, so that, via the treatment instrument insertion port, the fluorescence probe 52 is inserted through the treatment instrument insertion channel 51.

The fluorescence probe 52 has a proximal end to which the alignment light source 32 and the excitation light source 16 are connected, and is supplied with alignment light and excitation light. In addition, the fluorescence image pickup portion 21 is provided on the proximal end side of the fluorescence probe 52 such that the image of the fluorescence taken in by a probe distal end portion 52a is picked up by the fluorescence image pickup portion 21.

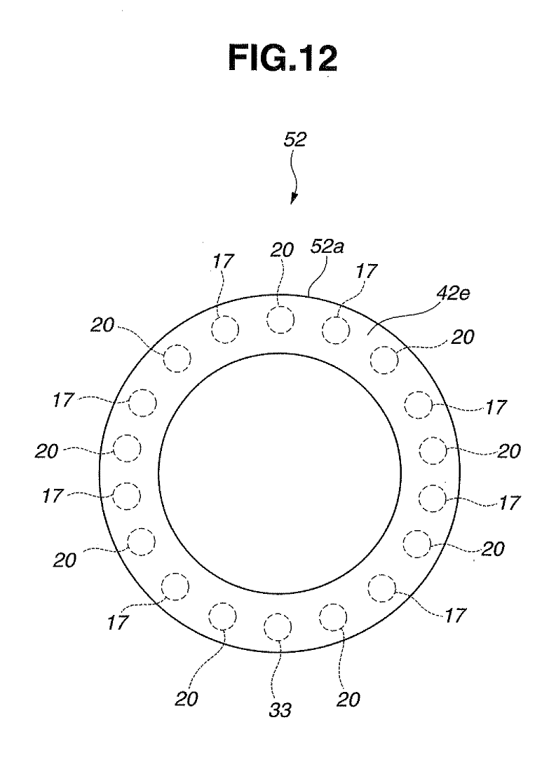

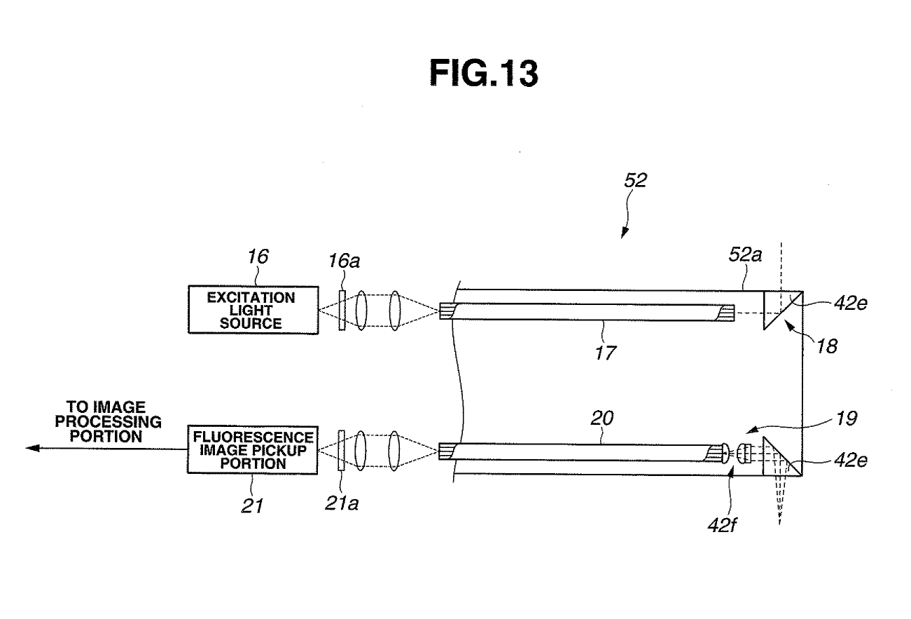

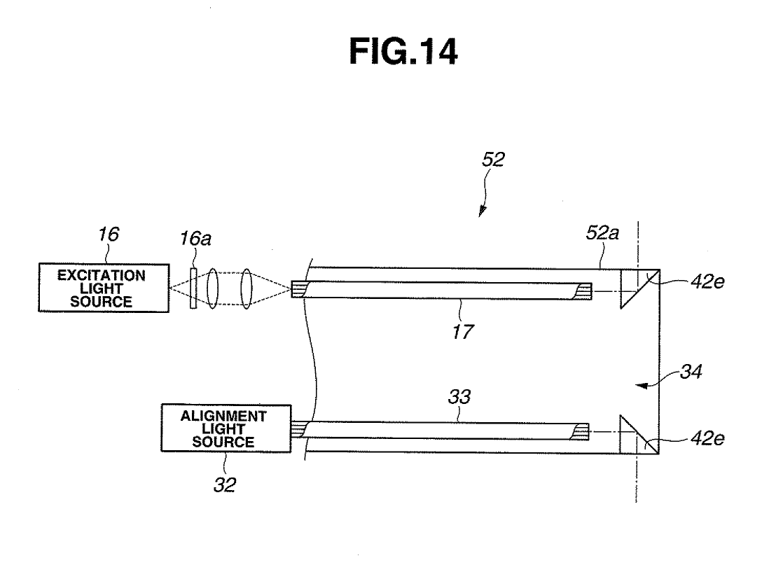

Next, a detailed description of the fluorescence probe 52 will be made with reference to FIGS. 12 to 14.

As shown in FIGS. 12 to 14, the fluorescence probe 52 has a plurality of the fluorescence image guides 20 and the excitation light guides 17 inserted therein along a circuit direction. In addition, the fluorescence probe 52 has one alignment light guide 33 inserted therethrough.

On the side surface of the probe distal end portion 52a of the fluorescence probe 52, a multi-purpose prism 42e covering a whole circumference of the distal end portion 2a is provided as a light deflecting portion. Here, the multi-purpose prism 42e directs the excitation light emitted from each of the excitation light guides 17 in a radially outward direction of the fluorescence probe 52, and directs the fluorescence entered from the side surface of the probe distal end portion 52a to one end side of each of the fluorescence image guides 20. That is, the multi-purpose prism 42e functions as a multi-purpose optical system serving as the light deflecting portion which is a part of the fluorescence optical system 19, the excitation optical system 18 as the excitation light-irradiating portion, and the alignment optical system 34.

Note that the prism may be disposed individually in plural numbers to each of the fluorescence image guides 20, excitation light guides 17, and the alignment light guide 33.

According to this, the fluorescence probe 52 is capable of irradiating, through the multi-purpose prism 42e, the excitation light emitted from the plurality of the excitation light guides 17 to the living tissue on the inner wall of the body cavity, from the periphery of the side surface of the probe distal end portion 52a, and also capable of taking in, through the multi-purpose prism 42e, the fluorescence from the lesioned part and the reflected light from the lesioned part and the normal tissue, from the periphery of the side surface of the probe distal end portion 52a. The fluorescence and the reflected light taken in by the multi-purpose prism 42e are image-formed by an image forming optical system 42f on end surfaces on a side closer to the probe distal end portion 52a of the fluorescence image guides 20.

In the endoscope apparatus 1E configured as such, as shown in FIG. 11, the endoscope insertion portion 2E is connected to the white light source 11 and the image processing apparatus 4, and after the fluorescence probe 52 is connected to the alignment light source 32, the excitation light source 16, and the image processing apparatus 4, the endoscope insertion portion 2E is inserted into the body cavity and the distal end portion 2a is guided to a target region. After the endoscope insertion portion 2E has reached the target region, fluorescence observation is performed with the endoscope apparatus 1E by inserting the fluorescence probe 52 through the treatment instrument insertion channel 51.

First, the operator administers a fluorescence material such as hematoporphyrin, for example, into a patient's body in advance. After a predetermined time period has passed, the fluorescence material is accumulated on the lesioned part. The operator inserts the endoscope insertion portion 2E into a body cavity of the patient to guide it to a target region.

At this time, in the endoscope insertion portion 2E, visible light such as white light supplied from the white light source 11 passes through the white light guide 12 to illuminate a living tissue in the body cavity via the white optical system 13. The endoscope insertion portion 2E takes in the reflected light from the illuminated living tissue as a subject image by the objective optical system 15a through the observation window 14, and picks up the subject image by the white light image pickup portion 15.

The white light image pickup portion 15 photoelectrically converts the subject image to generate an image pickup signal, and outputs the generated image pickup signal to the image processing apparatus 4. The image processing apparatus 4 processes the image pickup signal in the image processing portion 4a to generate a standard video signal, and outputs the video signal to the monitor 3 to display an endoscope image on the monitor 3.

The operator guides the distal end portion 2a of the endoscope insertion portion 2E to the target region in the body cavity while watching the display screen of the monitor 3. When the distal end portion 2a of the endoscope insertion portion 2E has reached the target region, the operator inserts the fluorescence probe 52 through the treatment instrument insertion channel 51 to perform fluorescence observation.

First, the operator inserts the fluorescence probe 52 through the treatment instrument insertion channel 51 of the endoscope insertion portion 2E such that the probe distal end portion 52a of the fluorescence probe 52 protrudes from an aperture of the channel by a predetermined distance.

Next, the operator irradiates inside the body cavity with the alignment light to confirm on the endoscope image the position of the multi-purpose prism 42e in the body cavity. In the fluorescence probe 52, the alignment light emitted from the alignment light source 32 passes through the alignment light guide 33 to be emitted into the body cavity via the multi-purpose prism 42e. The image of the reflected light of the alignment light from inside of the body cavity is picked up by the white light image pickup portion 15 to be displayed on the endoscope image. The operator confirms the position of the fluorescence optical system 19 using the alignment light displayed on the endoscope image.

The operator disposes the probe distal end portion 52a of the fluorescence probe 52 so as to be parallel with the inner wall of the body cavity. This makes it possible for the probe distal end portion 52a of the fluorescence probe 52 to take in from the multi-purpose prism 42e the fluorescence from the lesioned part existing on the inner wall of the body cavity and the reflected light from the lesioned part and the normal tissue at approximately the same distance.

Next, the operator supplies the excitation light to the fluorescence probe 52 to start fluorescence observation. The operator moves the probe distal end portion 52a of the fluorescence probe 52 forwardly and backwardly along the inner wall of the body cavity to perform fluorescence observation of the vicinity of the target region.

In the fluorescence probe 52, the excitation light emitted from the excitation light source 16 passes through the excitation light guides 17 via the excitation light filter 16a and is irradiated, via the multi-purpose prism 42e, to the living tissue on the inner wall of the body cavity from the periphery of the side surface of the probe distal end portion 52a.

When the lesioned part exists in the living tissue on the inner wall of the body cavity irradiated with the excitation light, the living tissue of the lesioned part (fluorescence material included therein) generates fluorescence. At this time, also the excitation light is irradiated to the lesioned part and the normal tissue to be reflected.

The fluorescence from the lesioned part and the reflected light from the lesioned part and the normal tissue are taken in via the multi-purpose prism 42e. The fluorescence and the reflected light taken in via the multi-purpose prism 42e are transmitted by the fluorescence image guides 20 and, via the barrier filter 21a, images thereof are respectively picked up by the fluorescence image pickup portion 21. This makes it possible for the fluorescence probe 52 to take in the fluorescence from the lesioned part existing on the inner wall of the body cavity and the reflected light from the normal tissue, from the periphery of the side surface of the probe distal end portion 52a, at approximately the same distance. Therefore, in the endoscope apparatus 1E, the intensity of the fluorescence from the lesioned part existing further from the probe distal end of the fluorescence probe 52 does not become smaller, compared with that of the leak light generated as a result that a part of the reflected light from the normal tissue existing nearer to the probe distal end of the fluorescence probe 52 passes through the barrier filter 21a.

The fluorescence image pickup portion 21 photoelectrically converts the respectively picked-up image of the fluorescence to generate an electrical signal, and outputs the generated electrical signal to the image processing apparatus 4.

The image processing apparatus 4 outputs to the image processing portion 4a the calculation data obtained by calculating the intensity of the fluorescence in the intensity calculation portion 4b based on the electrical signal from the fluorescence image pickup portion 21. The image processing portion 4a performs image display processing based on the calculation data from the intensity calculation portion 4b to output the video signal obtained by the image display processing to the monitor 3.

Similarly as described in FIG. 9, the image display processing is performed such that a graph is displayed to show the fluorescence intensity with respect to forward/backward movement distance (time period) of the distal end portion 2a for each of the plurality of fluorescence image guides 20.

Note that, though not shown, the fluorescence probe 52 may be the multi-purpose image guide 41 serving both as the excitation light guide 17 and the fluorescence image guide 20, similarly as described in FIG. 7.

As a result, in addition to obtaining the same effects as those in the embodiment 1, the endoscope apparatus 1E is capable of performing fluorescence observation in a duct in a body cavity where insertion of the endoscope insertion portion 2E is difficult, because the fluorescence probe 52 having a smaller diameter than that of the endoscope insertion portion 2E is used in the endoscope apparatus 1E.

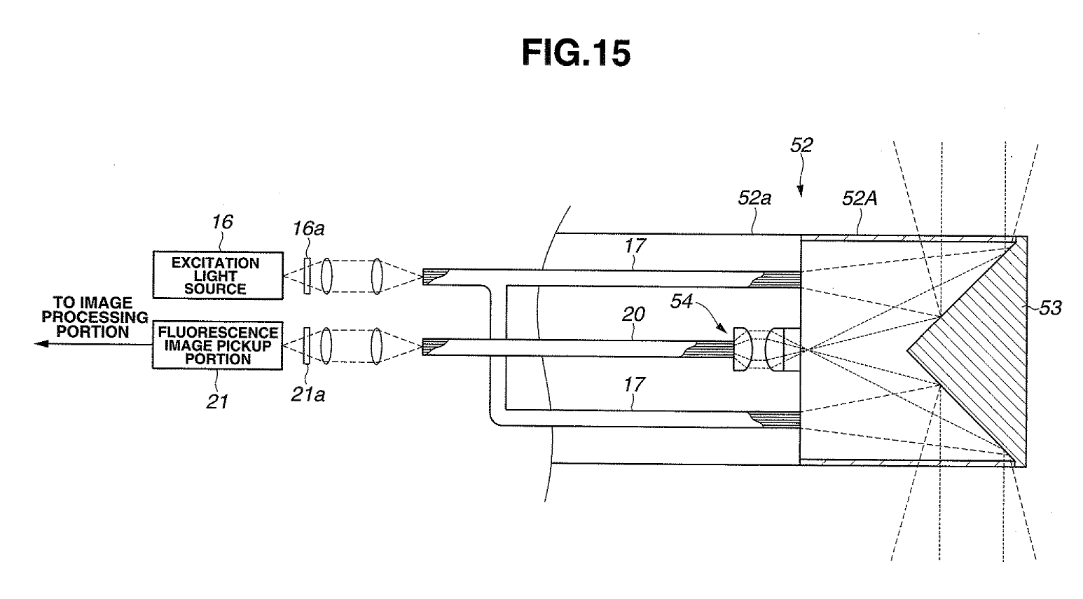

Note that the endoscope apparatus of the present embodiment may include the probe distal end portion 52a of the fluorescence probe 52 as modification examples to be described with reference to FIGS. 15 to 20.

The fluorescence probe 52 shown in FIG. 15 has the fluorescence image guide 20 at the center thereof and a plurality of excitation light guides 17 inserted around the fluorescence image guide 20.

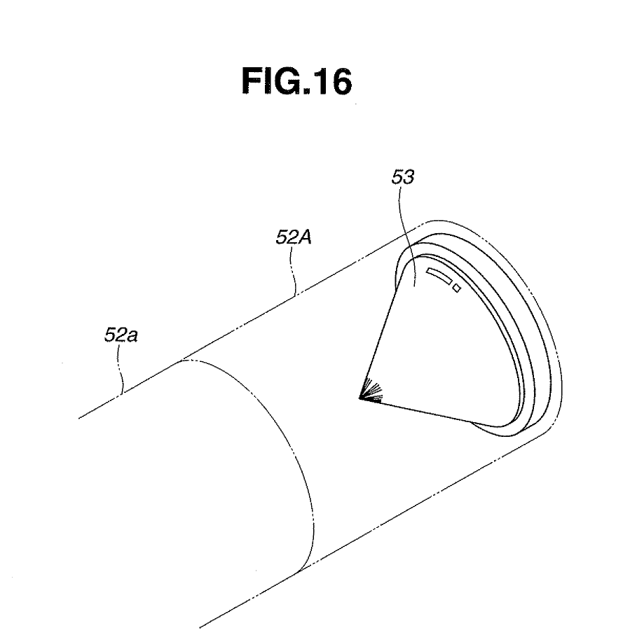

The probe distal end portion 52a of the fluorescence probe 52 has a circular ring-shaped transparent tube 52A provided over the whole circumference thereof. The transparent tube 52A has on a distal end opening surface a cone (conically-shaped) mirror 53 as a light deflecting portion, as shown in FIG. 16. The cone mirror 53 has a conically-shaped reflective surface on which light is reflected at a predetermined angle. That is, the cone mirror 53 functions not only as a circuit-direction irradiating portion (excitation light irradiating portion) for irradiating the excitation light emitted from each of the excitation light guides 17 toward the whole circumference in the radially outward direction of the probe 52, but also as a circuit-direction reflection portion for reflecting the fluorescence entered via the transparent tube 52A from the whole circumference in the radially outward direction of the probe 52 toward an end surface of the fluorescence image guide 20 on a side closer to the probe distal end portion 52a. Note that the shape of the cone mirror 53 may be a truncated conical shape instead of the conical shape.

An end surface of the image forming optical system 54 is disposed on the side closer to the probe distal end portion 52a of the fluorescence image guide 20 so as to face the cone mirror 53.

In the fluorescence probe 52 configured as such, the excitation light emitted from the excitation light source 16 passes through each of the excitation light guides 17 to be emitted from the end surface of each of the excitation light guides 17 on the side closer to the probe distal end portion 52a. The excitation light emitted from the end surface of each of the excitation light guides 17 is reflected on the conically-shaped surface of the cone mirror 53 in the transparent tube 52A to be directed in a radially outward direction of the fluorescence probe 52. Then, the excitation light passes through the transparent tube 52A to be irradiated to the living tissue on the inner wall of the body cavity over a whole circumferential direction of the fluorescence probe 52.

The fluorescence generated from the lesioned part on the inner wall of the body cavity is incident from the whole circumferential direction of the side surface of the probe distal end portion 52a and is reflected on the surface of the cone mirror 53 to be directed to the fluorescence image guide 20. At this time, also the excitation light reflected from the lesioned part and the normal tissue is directed similarly to the fluorescence image guide 20. Then, the fluorescence and the reflected light reflected from the cone mirror 53 are guided onto the end surface of the fluorescence image guide 20 on the side closer to the probe distal end portion 52a by the image forming optical system 19 and an image of the lesioned part is formed on the end surface.

The image-formed fluorescence and the reflected light are transmitted by the fluorescence image guide 20, and only the fluorescence passes through the barrier filter 21a and the image thereof is picked up by the fluorescence image pickup portion 21. Then, as described above, the picked-up image of the fluorescence and the leak light are converted into electrical signals by the photoelectrical conversion in the fluorescence image pickup portion 21, to be outputted to the image processing apparatus 4. The image processing apparatus 4 performs the signal processing as described above based on the signals from the fluorescence image pickup portion 21.

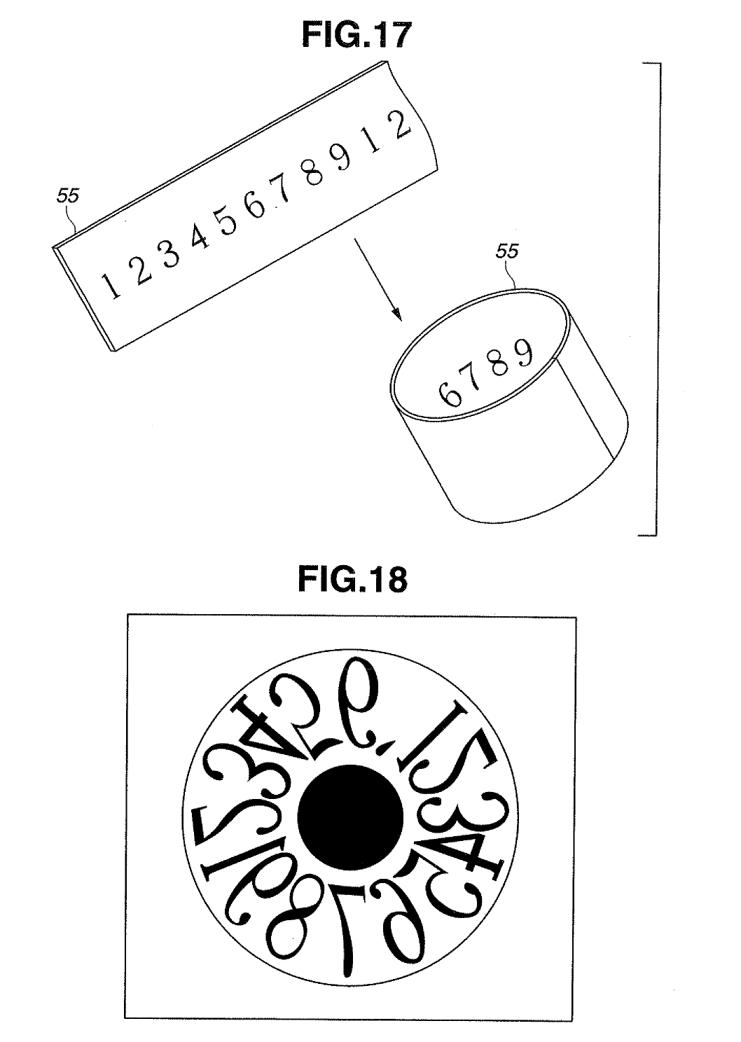

For example, as shown in FIG. 17, a strip-shaped test sample 55 on which numerical characters 1 to 9 are written is formed into a cylindrical shape and disposed on an outer circumferential surface of the transparent tube. At this time, a concentric image as shown in FIG. 18 is displayed on the monitor 3 by the image display processing.

The fluorescence probe 52 configured as describe above also allows fluorescence observation in a duct of a body cavity where insertion of the endoscope insertion portion is difficult, in addition to obtaining the same effects as those in the first embodiment, that is, the effects as described above.

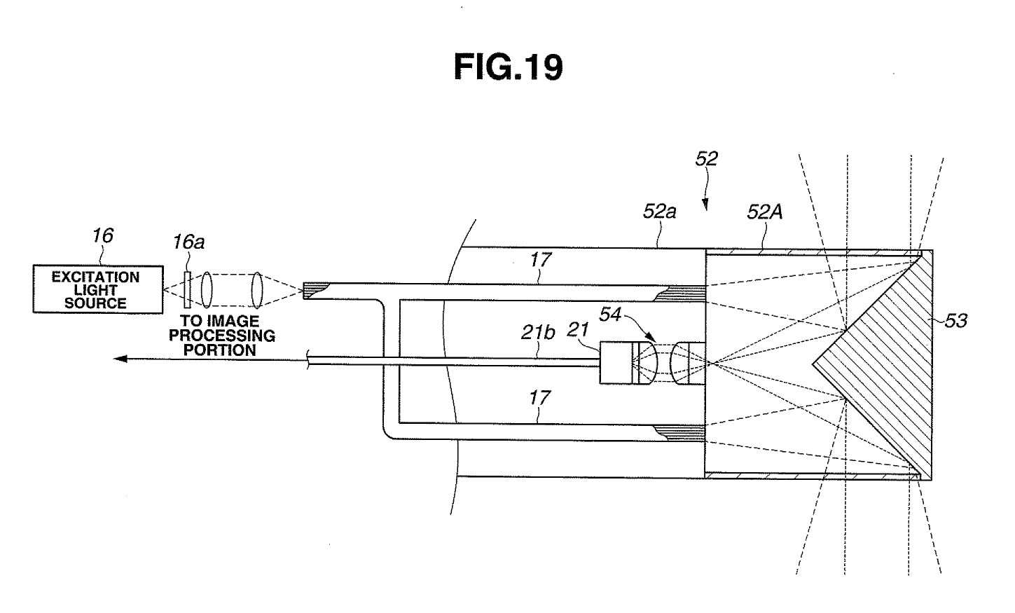

Note that, as shown in FIG. 19, the fluorescence image pickup portion 21 for outputting a generated image pickup signal to the image processing apparatus 4 (not shown) via the signal cable 21b may be used, instead of the fluorescence image guide 20. In this case, the image pickup element of the fluorescence image pickup portion 21 is disposed on an image-forming surface of the image forming optical system 54.

With such a configuration, the image pickup element of the fluorescence image pickup portion 21 picks up the image of the fluorescence generated in the lesioned part and the image of the excitation light reflected from the lesioned part and the normal tissue, to be converted into image pickup signals, and then the image pickup signals are outputted to the image processing apparatus 4 via the signal line 21b.

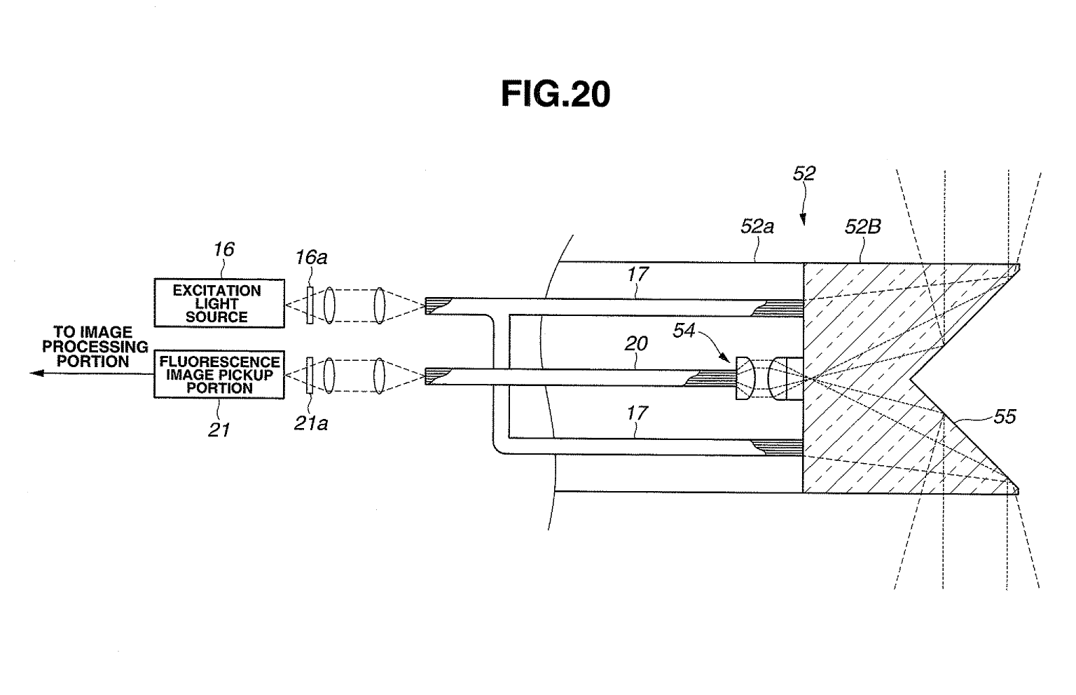

In addition, as shown in FIG. 20, instead of the transparent tube 52A and the cone mirror 53, there may be provided on a distal end side of the excitation light guides 17 and the image forming optical system 54 a transparent member 52B having a conically-shaped mirror surface 55 on the distal end side of the fluorescence probe 52.

The transparent member 52B is formed by conically shaving off one end surface of a solid transparent columnar member, and inside of the conically shaved off mirror surface 55 serves as a reflective surface. In this case, the transparent member 52B reflects the excitation light, fluorescence, and reflected light on the conically shaped mirror surface 55.

With such a configuration, the probe distal end portion 52a can be formed with a simple configuration.

It is apparent that various modifications are possible based on the present invention without departing from the spirit and scope of the invention. The present invention is not limited by a specific embodiment but only limited by the appended claims.

What is claimed is:

1. An endoscope apparatus provided with an endoscope insertion portion to be inserted into a body cavity, capable of performing fluorescence observation, the endoscope apparatus comprising:
a transferring portion for transferring information on an image of a region to be examined to a proximal end side of the endoscope insertion portion, the transferring portion being provided to the endoscope insertion portion;
a light deflecting portion for directing fluorescence entered from a side surface of a distal end portion of the endoscope insertion portion to one end side of the transferring portion; and
an image forming optical system for image-forming fluorescence from the light deflecting portion at one end of the transferring portion.

2. The endoscope apparatus according to claim 1, wherein the light deflecting portion is a circuit-direction reflection portion for reflecting fluorescence entered via the side surface of the distal end portion from a whole circumference in a radially outward direction of the endoscope insertion portion to the one end side of the transferring portion.

3. The endoscope apparatus according to claim 2, wherein the circuit-direction reflection portion is a conically-shaped mirror.

4. The endoscope apparatus according to claim 2, wherein the circuit-direction reflection portion is a conically-shaped prism.

5. The endoscope apparatus according to claim 1, wherein the transferring portion includes an image pickup portion at one end thereof; and the transferring portion is a signal line for transferring image data obtained by the image pickup portion to a proximal end side of the endoscope insertion portion.

6. The endoscope apparatus according to claim 2, wherein the transferring portion includes an image pickup portion at one end thereof, and the transferring portion is a signal line for transferring image data obtained by the image pickup portion to a proximal end side of the endoscope insertion portion.

7. The endoscope apparatus according to claim 1, wherein the transferring portion is an optical fiber bundle for transferring a fluorescence image formed by the image forming optical system to a proximal end side of the endoscope insertion portion.

8. The endoscope apparatus according to claim 2, wherein the transferring portion is an optical fiber bundle for transferring a fluorescence image formed by the image forming optical system to a proximal end side of the endoscope insertion portion.

9. The endoscope apparatus according to claim 1, wherein the endoscope insertion portion includes an excitation light irradiating portion for irradiating excitation light from a side surface on a distal end side of the endoscope insertion portion and a fluorescence filter for transmitting fluorescence generated from the region to be examined by the excitation light and shutting off other light.

10. The endoscope apparatus according to claim 2, wherein the endoscope insertion portion includes an excitation light irradiating portion for irradiating excitation light from a side surface on a distal end side of the endoscope insertion portion and a fluorescence filter for transmitting fluorescence generated from the region to be examined by the excitation light and shutting off other light.

11. The endoscope apparatus according to claim 9, wherein the excitation light irradiating portion includes a circuit-direction irradiating portion for irradiating the excitation light to a whole circumference in a radially outward direction of the endoscope insertion portion.

12. The endoscope apparatus according to claim 10, wherein the excitation light irradiating portion includes a circuit-direction irradiating portion for irradiating the excitation light to the whole circumference in the radially outward direction of the endoscope insertion portion.

13. The endoscope apparatus according to claim 3, wherein the circuit-direction reflection portion further includes a function for irradiating excitation light to the whole circumference in the radially outward direction of the endoscope insertion portion, and the endoscope insertion portion is provided with a fluorescence filter for transmitting fluorescence generated from the region to be examined by the excitation light and shutting off other light.

14. An endoscope probe provided with a probe insertable into a treatment instrument insertion channel of the endoscope insertion portion to be inserted into a body cavity, capable of performing fluorescence observation, the endoscope probe comprising:
a transferring portion for transferring information on an image of a region to be examined to a proximal end side of the probe, the transferring portion being provided to the probe;
a light deflecting portion for directing fluorescence entered from a side surface of a distal end portion of the probe to one end side of the transferring portion; and
an image forming optical system for image-forming fluorescence from the light deflecting portion at one end of the transferring portion.

15. The endoscope probe according to claim 14, wherein the light deflecting portion is a circuit-direction reflection portion for reflecting the fluorescence entered from a whole circumference in a radially outward direction of the probe via the side surface of the distal end portion to the one end side of the transferring portion.

16. The endoscope probe according to claim 14, wherein the probe includes an excitation light irradiating portion for irradiating excitation light from the side surface of the distal end portion, and a fluorescence filter for transmitting fluorescence generated from the region to be examined by the excitation light and shutting off other light.

17. The endoscope probe according to claim 15, wherein the probe includes an excitation light irradiating portion for irradiating excitation light from the side surface of the distal end portion and a fluorescence filter for transmitting the fluorescence generated from the region to be examined by the excitation light and shutting off other light.

18. An endoscope apparatus comprising:
the endoscope probe as recited in claim 14; and
an endoscope insertion portion for irradiating white light to a region to be examined and receiving the white light reflected to be returned from the region to be examined.

19. An endoscope apparatus comprising:
the endoscope probe as recited in claim 15; and
an endoscope insertion portion for irradiating white light to a region to be examined and receiving the white light reflected to be returned from the region to be examined.

20. An endoscope apparatus comprising:
the endoscope probe as recited in claim 16; and
an endoscope insertion portion for irradiating white light to a region to be examined and receiving the white light reflected to be returned from the region to be examined.

21. The endoscope apparatus according to claim 1, further comprising a position indicating portion for indicating a position of the light deflecting portion in the distal end portion of the endoscope insertion portion.

22. The endoscope apparatus according to claim 21, wherein the position indicating portion is an alignment light irradiating portion provided in the distal end portion of the endoscope insertion portion.

23. The endoscope apparatus according to claim 21, wherein the position indicating portion is a marking displayed on an endoscope image.

24. An endoscope apparatus provided with an endoscope insertion means to be inserted into a body cavity, capable of performing fluorescence observation, the endoscope apparatus comprising:
transferring means for transferring information on an image of a region to be examined to a proximal end side of the endoscope insertion means;
light deflecting means for directing fluorescence entered from a side surface of a distal end portion of the endoscope insertion means to one end side of the transferring means; and
image forming optical means for image-forming fluorescence from the light deflecting means at one end of the transferring means.

25. An endoscope probe provided with a probe insertable into a treatment instrument insertion channel of an endoscope insertion portion to be inserted in a body cavity, capable of performing fluorescence observation, the endoscope probe comprising:
transferring means for transferring information on an image of a region to be examined to a proximal end side of the probe, the transferring means being provided to the probe;
light deflecting means for directing fluorescence entered from a side surface of a distal end portion of the probe to one end side of the transferring means; and
image forming optical means for image-forming fluorescence from the light deflecting means at one end of the transferring means.
